(19) United States
(12) Patent Application Publication
(10) Pub. No.: US 2026/0014354 A1
(43) Pub. Date: Jan. 15, 2026

(54) Universal Tunneler

(71) Applicant: Bard Peripheral Vascular, Inc., Franklin Lakes, NJ (US)

(72) Inventor: Mark S. Kelemen, Woods Cross, UT (US)

(21) Appl. No.: 18/993,987

(22) PCT Filed: Jul. 26, 2022
(86) PCT No.: PCT/US22/38380

§ 371 Date: Jan. 13, 2025

(51) Int. Cl. A61M 25/01; A61B 17/32; A61M 25/06

(52) U.S. Cl. CPC A61M 25/0194; A61B 17/32; A61M 25/0662; A61B 2017/320056

(57) ABSTRACT

Embodiments disclosed herein are directed to a universal tunneler (100) including a shaft (110) and a hub. The hub (130) includes an “H-shaped” grip piece (140) configured to engage an end portion of the shaft and further includes a sheath (152) slidably engaged therewith. Sliding the sheath longitudinally causes the gripping arms (146A, 146B) to deflect radially inwards and grip a tip portion of the catheter (28) disposed therebetween. The tunneler can then be urged through a subcutaneous tunnel pulling the catheter therewith. Advantageously, the hub does not require the catheter tip to include any specialized engagement structures. Further, the hub grips and encloses the tip structures mitigating any damage during the tunneling process. The “HI-shaped” grip portion provides a simplified structure for case of manufacture and reducing costs.

### SUMMARY

Briefly summarized, embodiments of the present invention are directed to a universal tunneler including a shaft and a hub coupled thereto. The hub includes an “H-shaped” grip piece configured to engage an end of the shaft and further includes a sheath slidably engaged with a portion of the grip. Sliding the sheath longitudinally causes the gripping arms of the grip to deflect radially inwards to grasp a tip portion of the catheter disposed therebetween. The tunneler can then be urged through a subcutaneous tunnel, pulling the catheter therewith. Advantageously, the hub does not require any specialized engagement structures formed as part of the catheter tip in order to engage with the tip. Further the hub grips the tip portion at a position that is spaced apart from the tip structures mitigating any damage thereto. Further still, the hub encloses the tip structures of the catheter to protect the tip structures from any damage during the tunneling process. The “H-shaped” grip portion also provides a simplified structure for ease of manufacture, reducing associated costs.

Disclosed herein is a tunneler configured to place a portion of a catheter within a subcutaneous tunnel and including, a shaft extending along a central longitudinal axis, and having a hub disposed at a first end and having, a grip coupled to an end portion of the shaft and including a cross-beam supporting a first gripping arm and a second gripping arm, each of the first gripping arm and the second gripping arm transitionable between an unlocked position and a locked position, and a sheath extending annularly about a portion of the grip and slidably engaged therewith along the central longitudinal axis to transition the first gripping arm and the second gripping arm between the unlocked position and the locked position.

In some embodiments, the first gripping arm extends substantially longitudinally from a first end of the cross-beam and the second gripping arm extends substantially longitudinally from a second end of the cross-beam, opposite the first end of the cross-beam, the cross-beam extending perpendicular to the central longitudinal axis.

In some embodiments, each of the first gripping arm and the second gripping arm includes an inner surface and an outer surface, in the unlocked position the outer surface is angled relative to the central longitudinal axis and the inner surface extends parallel to the central longitudinal axis, and in the locked position the inner surface is angled relative to the central longitudinal axis and the outer surface extends parallel to the central longitudinal axis.

In some embodiments, in the unlocked position the inner surfaces of the first gripping arm and the second gripping arm define an inner diameter (d2) that is equal to, or greater than, an outer diameter of the catheter.

In some embodiments, in the unlocked position the outer surface of each of the first gripping arm and the second gripping arm extends from an outer diameter (d3) of the cross-beam to an outer diameter (d4) of a tip of the gripping arm, the outer diameter (d) at the tip of the gripping arm being larger than the outer diameter (d3) of the cross-beam.

In some embodiments, an inner diameter of the sheath is equal to, or greater than the outer diameter (d3) of the cross-beam and is less than the outer diameter (d4) of the tip of the gripping arm.

In some embodiments, in the locked position the inner surface of each of the first gripping arm and the second gripping arm extends from an inner diameter (d2) adjacent the cross-beam, to an inner diameter (d5) adjacent the tip of the gripping arm that is less than the outer diameter of the catheter.

In some embodiments, wherein one or both of the first gripping arm and the second gripping arm includes a beveled surface adjacent a tip thereof and angled radially inwards towards the central longitudinal axis.

In some embodiments, one of the first gripping arm and the second gripping arm is biased towards the unlocked position.

In some embodiments, the grip is formed of a substantially rigid, or resilient, material such as a plastic, polymer, metal, alloy, or composite.

In some embodiments, the tunneler further includes a handle disposed at a second end of the shaft, the handle including an actuator configured to slide the sheath longitudinally between a first position and a second position and transition the hub between a locked position and an unlocked position.

In some embodiments, the grip further includes a first shaft arm extending longitudinally from the first end of the cross-beam, and a second shaft arm extending longitudinally from the second end of the cross-beam, the first shaft arm and the second shaft arm engaging the end portion of the shaft in one of a press-fit, snap-fit, or interference fit engagement.

In some embodiments, the grip further includes a third gripping arm disposed opposite a fourth gripping arm across the central longitudinal axis, each of the third gripping arm and the fourth gripping arm extending longitudinally and transitionable between an unlocked position and a locked position.

Also disclosed is a method of placing a portion of a catheter within a subcutaneous tunnel including, urging a tunneler subcutaneously between a first incision and a second incision, the tunneler including a hub having a grip coupled to an end portion of a shaft of the tunneler, and a sheath extending annularly about a portion of the grip and slidably engaged therewith, placing a tip portion of the catheter between a first gripping arm and a second gripping arm of the grip, sliding the sheath between a first position and a second position, elastically deflecting the first gripping arm and the second gripping arm radially inwards to engage the tip portion of the catheter, and urging the hub and tip portion assembly through the subcutaneous tunnel.

In some embodiments, the sheath in the second position encloses the tip portion of the catheter mitigating damage thereto.

In some embodiments, urging the hub of the tunneler subcutaneously between the first incision and the second incision further includes forming the subcutaneous tunnel.

In some embodiments, the method further includes actuating an actuator mechanism to transition the sheath between the first position and the second position, the actuator mechanism disposed on a handle of the tunneler.

In some embodiments, the catheter is one of a single lumen catheter, dual lumen catheter, multi-lumen catheter, a central venous catheter (CVC), a peripherally inserted central catheter (PICC), or a hemodialysis catheter.

Also disclosed is a method of making a tunneler for placing a portion of a catheter within a subcutaneous tunnel including, forming a shaft extending along a central longitudinal axis, coupling a hub at a first end of the shaft, including coupling a grip to an end portion of the shaft, the grip including a cross-beam supporting a first gripping arm and a second gripping arm, each transitionable between an unlocked position and a locked position, and slidably engaging a sheath with a portion of the grip, the sheath transitionable between a first position and a second position to transition the first gripping arm and the second gripping arm between the unlocked position and the locked position.

In some embodiments, the first gripping arm extends longitudinally from a first end of the cross-beam and the second gripping arm extends longitudinally from a second end of the cross-beam, opposite the first end of the cross-beam, the cross-beam extending perpendicular to the central longitudinal axis.

In some embodiments, each of the first gripping arm and the second gripping arm includes an inner surface and an outer surface, in the unlocked position the outer surface is angled relative to the central longitudinal axis and the inner surface extends parallel to the central longitudinal axis, and in the locked position the inner surface is angled relative to the central longitudinal axis and the outer surface extends parallel to the central longitudinal axis.

In some embodiments, in the unlocked position the inner surfaces of the first gripping arm and the second gripping arm define an inner diameter (d2) that is equal to, or greater than, an outer diameter of the portion of the catheter.

In some embodiments, in the unlocked position the outer surface of each of the first gripping arm and the second gripping arm extends from an outer diameter (d3) of the cross-beam to an outer diameter (d4) at a tip of the gripping arms, the outer diameter (d) at the tip of the gripping arms being larger than the outer diameter (d3) of the cross-beam.

In some embodiments, an inner diameter of the sheath is equal to, or greater than, the outer diameter (d3) of the cross-beam and is less than the outer diameter (d4) of the tip of the gripping arms.

In some embodiments, in the locked position the inner surface of each of the first gripping arm and the second gripping arm extends from an inner diameter (d2) adjacent the cross-beam, to an inner diameter (d5) adjacent the tip of the gripping arms that is less than the outer diameter of the portion of the catheter.

In some embodiments, one or both of the first gripping arm and the second gripping arm includes a beveled surface adjacent a tip thereof and angled radially inwards towards the central longitudinal axis.

In some embodiments, one of the first gripping arm and the second gripping arm is biased towards the unlocked position.

In some embodiments, the grip is formed of a substantially rigid, or resilient, material such as a plastic, polymer, metal, alloy, or composite.

In some embodiments, the method further includes a handle disposed at a second end of the shaft and including an actuator configured to slide the sheath longitudinally between the first position and the second position and transition the hub between a locked position and an unlocked position.

In some embodiments, the grip further includes a first shaft arm extending longitudinally from the first end of the cross-beam, and a second shaft arm extending proximally from the second end of the cross-beam, the first shaft arm and the second shaft arm engaging the end portion of the shaft in one of a press-fit, snap-fit, or interference fit engagement.

In some embodiments, the grip further includes a third gripping arm disposed opposite a fourth gripping arm across the central longitudinal axis, each of the third gripping arm and the fourth gripping arm extending longitudinally and transitionable between an unlocked position and a locked position.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of an exemplary vascular access device for use with a universal tunneler, in accordance with embodiments disclosed herein.

FIG. 2 shows a perspective view of a universal tunneler, in accordance with embodiments disclosed herein.

FIGS. 3A-3C shows an exemplary method of use for a universal tunneler, in accordance with embodiments disclosed herein.

FIG. 4A shows a longitudinal cross-section view of a hub of a universal tunneler in an unlocked position, in accordance with embodiments disclosed herein.

FIG. 4B shows a longitudinal cross-section view of a hub of a universal tunneler in a locked position, in accordance with embodiments disclosed herein.

FIG. 5 shows a perspective view of a grip portion for a hub of a universal tunneler, in accordance with embodiments disclosed herein.

FIG. 6 shows a lateral cross-section view of a hub of a universal tunneler, in accordance with embodiments disclosed herein.

FIG. 7 shows a lateral cross-section view of a hub of a universal tunneler, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

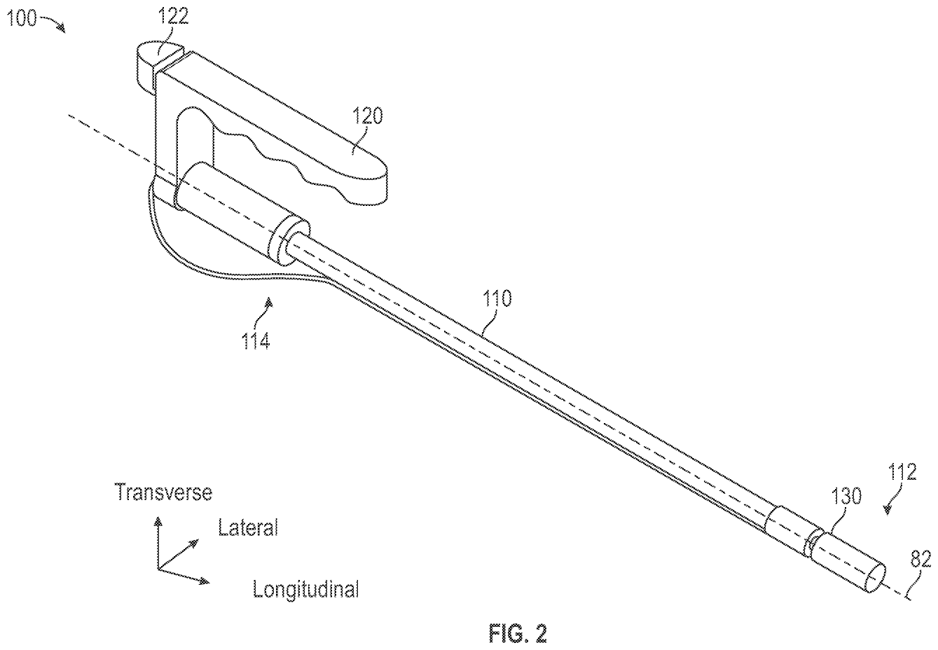

To assist in the description of embodiments described herein, as shown in FIG. 2, a longitudinal axis extends substantially parallel to an axial length of the tunneler. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

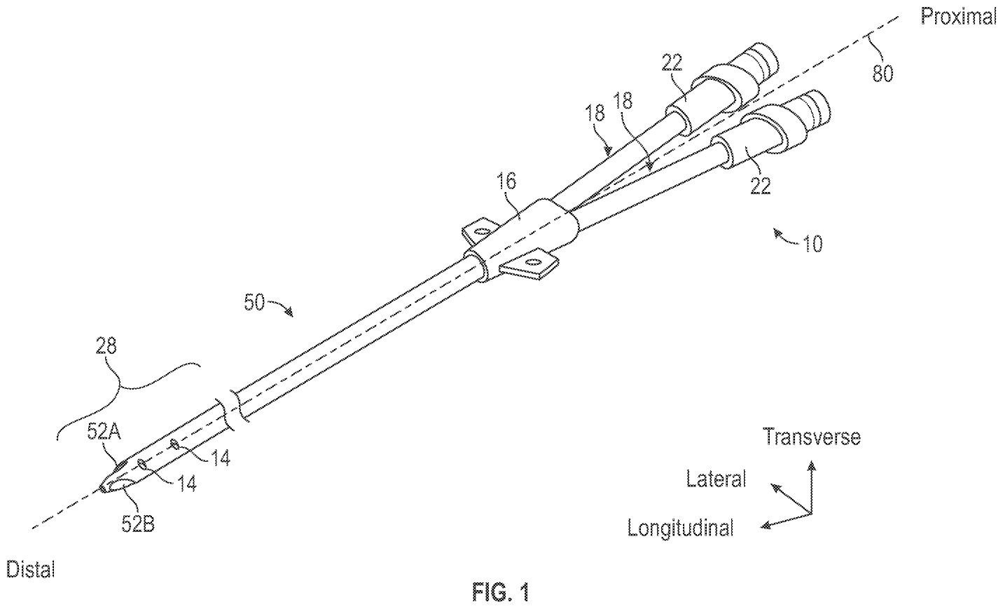

FIG. 1 shows an exemplary vascular access device, or catheter 10, for use with exemplary embodiments disclosed herein. Exemplary catheters 10 can include single lumen catheters, dual lumen catheters multi-lumen catheters, central venous catheter (CVC), peripherally inserted central catheter (PICC), hemodialysis catheters, or the like. The catheter 10 can generally include a catheter body 50 extending along a central longitudinal axis 80 and defining one or more lumen 52, e.g. a first lumen 52A and a second lumen 52B. Each lumen 52 can communicate with an opening disposed at a distal tip of the catheter 10. Optionally, additional openings 14 can be disposed in a side wall of the catheter body 50 and can communicate with the lumen 52.

In an embodiment, a proximal end of the catheter body 50 can be supported by a bifurcation 16. The catheter 10 can further include one or more extension legs 18 extending proximally from the bifurcation 16. Each extension leg 18 can be in fluid communication with a lumen 52 of the catheter body 50. The catheter 10 can further include a coupling 22 disposed at a proximal end of the extension leg 18 and configured to couple with a fluid line, syringe or similar device to provide fluid communication therebetween. Exemplary couplings can include spin nut, bayonet couplings, luer locks, or the like.

In an embodiment, the catheter 10 can include a tip portion 28 disposed distally and configured to be placed within a vasculature of a patient 90. In an embodiment, a proximal end of the catheter 10, e.g. bifurcation 16, extension leg(s) 18, etc. can be configured to remain outside of the patient's body 90. In an embodiment, the tip portion 28 can include various openings, apertures, split tip(s), fluid flow structures, lumen morphology, valves, or the like, depending on the specific use of the catheter. The tip portion 28, including these various structures, can also be delicate and easily damaged during placement which in turn can affect the flow characteristics and the efficacy of the catheter device 10.

FIG. 2 shows an embodiment of a universal tunneler (“tunneler”) 100 configured to place a catheter 10, or similar device, within a patient 90. The tunneler 100 can generally include a shaft 110 extending along a central longitudinal axis 82 between a first end 112 and a second end 114. The tunneler 100 can include an engagement hub (“hub”) 130 disposed at the first end 112, configured to engage a tip structure 28 of a catheter 10, as described in more detail herein.

In an embodiment, the tunneler 110 can include a blunt tip disposed at the second end 114 and can be configured to be urged subcutaneously through a subcutaneous tunnel 60, as described in more detail herein. In an embodiment, the tunneler 100 can include a handle 120 disposed at the second end 114 and can be configured to be grasped by a user to manipulate the tunneler 100. In an embodiment, the handle 120 can include an actuator 122, or similar mechanism, configured to actuate the hub 130 and transition the hub 130 between an unlocked position and a locked position, as described in more detail herein.

In an embodiment, the hub 130 of the universal tunneler 100 is configured to grasp various configurations of a tip portion 28 of a catheter 10 and tunnel the tip portion 28 subcutaneously. Once tunneled, the tip portion 28 can be placed within the vasculature of the patient 90. Advantageously, the hub 130 of the tunneler 100 does not require the tip portion 28 of the catheter 10 to include any specific engagement structures in order to engage the tip portion 28. As such, the tunneler 100 can be used with a variety of catheters and tip designs. Further, the universal tunneler 100 can grasp and enclose the tip portion 28 protecting the tip portion 28 during tunneling, mitigating damage thereto.

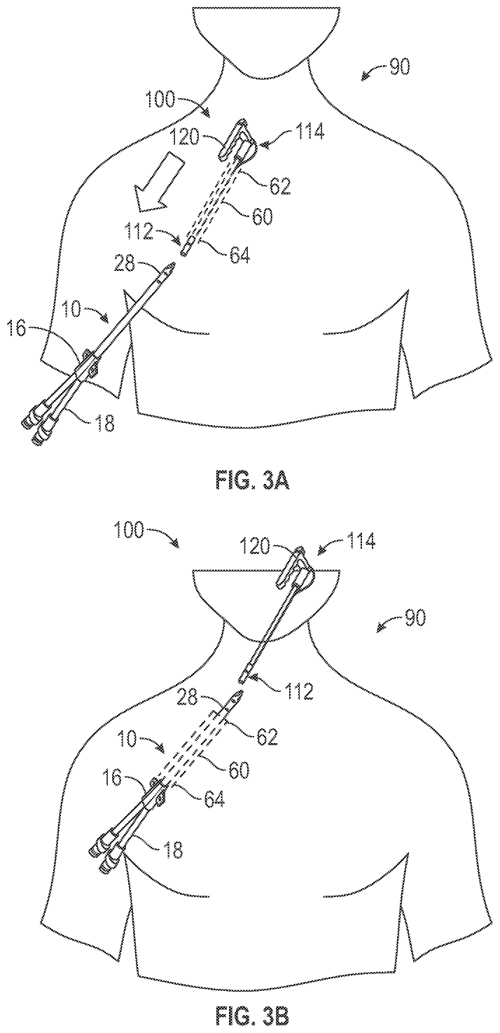

FIGS. 3A-3C show an exemplary method of placing a catheter 10, such as a CVC, hemodialysis catheter, or the like, using a universal tunneler 100. Initially, incisions are made at a first position, e.g. position “A” 62, adjacent a collar bone area, and a second position, e.g. position “B” 64, adjacent a pectoral area. A tunneler 100, or similar device, can be urged subcutaneously between the position “A” 62 and position “B” 64 to form a subcutaneous tunnel 60.

In an embodiment, the subcutaneous tunnel 60 can be formed by the tunneler 100. In an embodiment, the subcutaneous tunnel 60 can be formed by a separate device from that of the tunneler 100. In an embodiment, the tunneler 100 can include a tunneling bit structure (not shown) coupled with the hub 130 to form the tunnel 60. Once the tunnel 60 has been formed, the bit structure can be removed to allow the hub 130 to be coupled with the catheter 10.

In an embodiment, the hub 130 of the tunneler 100 can emerge from the incision at the second position 64 and can be coupled with a tip portion 28 of the catheter 10, as described in more detail herein. With the tip portion 28 of the catheter 10 coupled with the hub 130 of the tunneler 100, the tunneler 100 can be withdrawn through the subcutaneous tunnel 60 from the second position 64 to the first position 62. Advantageously, the hub 130 can enclosed the tip portion 28 to mitigate damage to the tip portion 28 during tunneling. Further, the hub 130 is configured to engage a various structures of tip portions 28 and does not require any specific engagement structures formed as part of the tip portion 28.

In an embodiment the tunneler 100, including a blunt tip at the second end 114 instead of the handle 120, can be urged subcutaneously to form the subcutaneous tunnel 60. For example, a tip 28 of the catheter 10 can be engaged with the hub 130 at the first end 112 of the tunneler 100, as described herein. The second end 114 can be inserted through an incision at position “B” 64 and urged subcutaneously to position “A” 62, forming the subcutaneous tunnel 60 therebetween. A user can then grasp the second end 114 emerging from the incision at position “A” 62 and pull the tunneler 110 through the subcutaneous tunnel 60 until the tip 28 of the catheter 10 extends from the incision at position “A” 62 (FIG. 3B).

As shown in FIG. 3C, with the tip portion 28 tunneled subcutaneously to position “A” 62, the hub 130 of the tunneler 100 can be disengaged from the tip portion 28 of the catheter 10, and the tip portion 28 can be reinserted through the incision at position “A” 62 and placed within the vasculature of the patient 90.

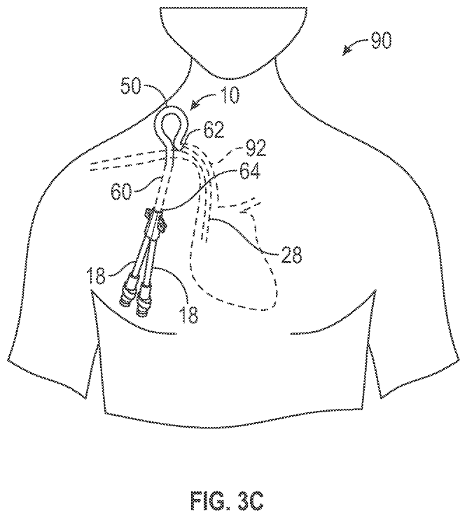

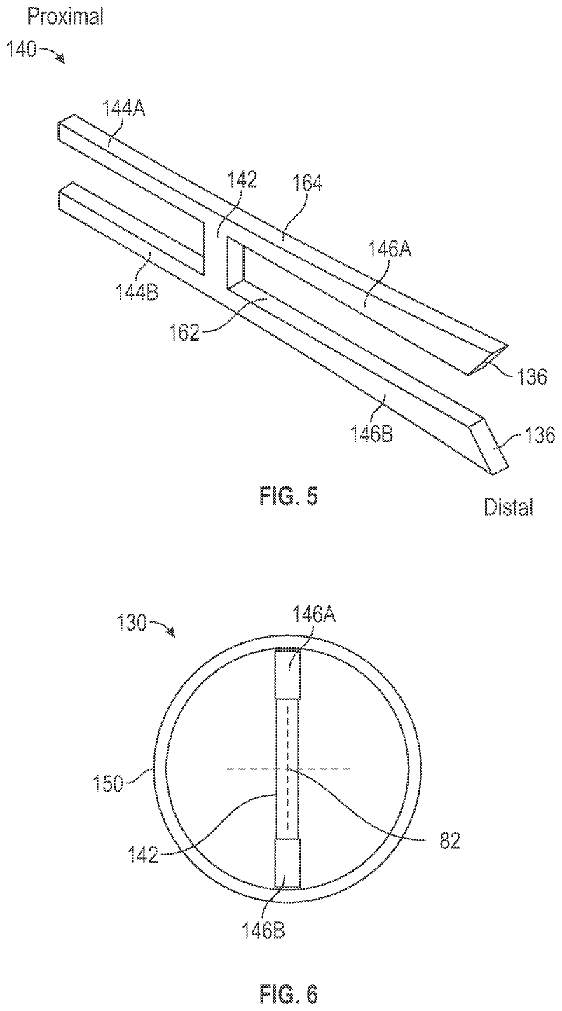

FIGS. 4A-6 show further details of an embodiment of the tunneler hub 130. FIG. 4A shows a longitudinal cross-sectional view of the hub 130 in an unlocked, or open position, and FIG. 4B shows a cross-sectional view of the hub 130 in a locked, or closed position. The hub 130 can generally include a grip 140 and a sheath 150. The grip 140 can be coupled with a distal end of the tunneler shaft 110 using one or more of a press-fit, snap-fit, interference fit, or threadable engagement. Alternatively, or in addition to, the grip 140 can be adhered, bonded, welded, ultrasonically welded, or the like, to the tip of the shaft 110. In an embodiment, the grip 140 can be formed of a substantially rigid, or resilient, material such as a plastic, polymer, metal, alloy, composite, or the like.

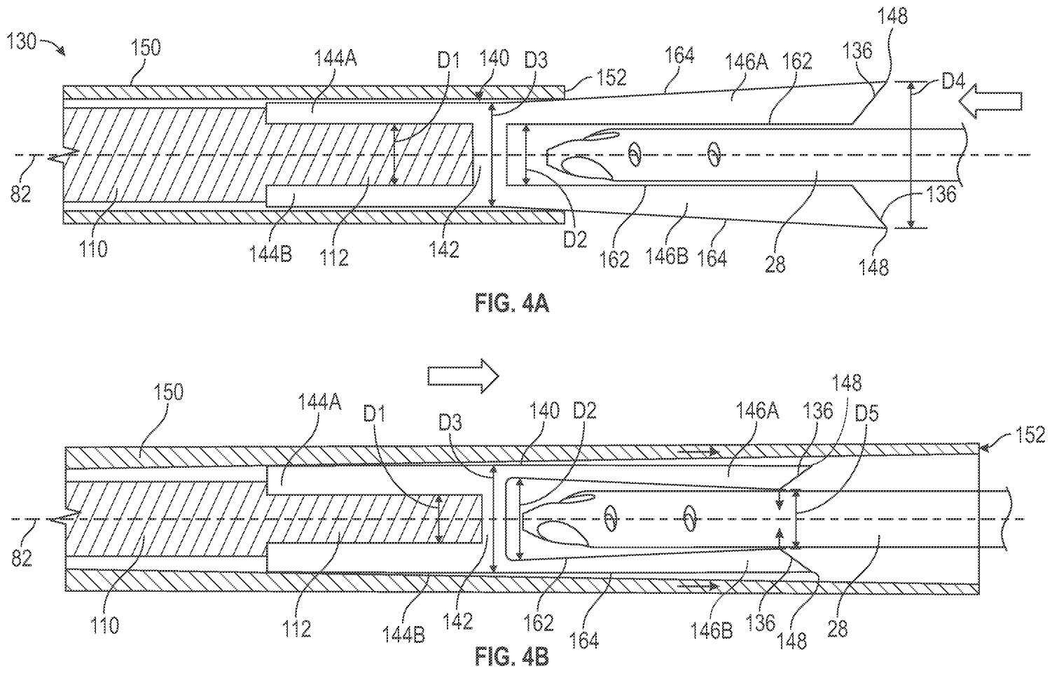

In an embodiment, as shown in FIG. 5, the grip 140 can substantially define an “H-shaped” structure, having a central cross-beam 142, one or more shaft arms 144 extending in a first direction from the cross-beam 142 and one or more gripping arms 146 extending in a second direction, opposite the first direction, from the cross-beam 142. Advantageously, the “H-shaped” structure can provide a simplified manufacturing design, improving manufacturing efficiencies and reducing costs.

In an embodiment, the grip 140 can include a first shaft arm 144A extending from a first end of the cross beam 142 and a second shaft arm 144B extending from a second end of the cross beam 142, opposite the first end of the cross beam 142. A first end 112 of the shaft 110 can extend between the first shaft arm 144A and the second shaft arm 144B to secure the grip 140 thereto. In an embodiment, a first diameter (dl) between the first shaft arm 144A and the second shaft arm 144B can be equal to, or slightly less than, a diameter of the first end 112 of the shaft 110 to engage the shaft 110 in a press-fit or interference fit engagement. In an embodiment, the shaft 110 and/or shaft arms 144 can include one or more grooves, abutments, pawls, barbs, or similar structures configured to engage the grip 140 with the shaft 110 in a press-fit, interference fit, or snap-fit engagement.

In an embodiment, the grip 140 can include a first gripping arm 146A extending from the first end of the cross beam 142 and a second gripping arm 146B extending from the second end of the cross beam 142, opposite the first end of the cross beam 142. The first gripping arm 146A and the second gripping arm 146B can be flexible and can elastically deform between an open position (FIG. 4A) where the tips 148 of the first gripping arm 146A and the second gripping arm 146B are disposed radially outwards, and a closed position (FIG. 4B) where the distal ends of the first gripping arm 146A and the second gripping arm 146B are disposed radially inwards.

As shown in FIG. 4A, in the open position, an inner surface 162 of the gripping arms 146 can extend substantially parallel to each other, and parallel to a central longitudinal axis 82, and can define a second diameter (d2) therebetween. The second diameter (d2) can be equal to, or larger than an outer diameter of the tip portion 28 of the catheter 10. Further, an outer surface 164 of the gripping arms 146 can extend at a shallow angle relative to a central longitudinal axis 82 of the tunneler 100 from an outer diameter of the cross-beam (d3) to an outer diameter (d4) at a tip 148 of the gripping arms 146. The outer diameter (d4) at the tip 148 of the gripping arm 146 being larger than the outer diameter of the cross-beam (d3).

The hub 130 can further include a sheath 150 extending annually about a distal portion of the shaft 110 and a portion of the grip 140. The inner diameter of the sheath 150 can be equal to, or slightly larger than the outer diameter (d3) of the cross-beam 142 but less than the outer diameter (d4) at the tip 148 of gripping arm 146 in the open position. In an embodiment, the sheath 150 can define a continuous inner diameter extending over a longitudinal axis. In an embodiment, at least a portion of the inner diameter of sheath 150 can be tapered, i.e. defining a continuous change in diameter between a first inner diameter and a second inner diameter. The second inner diameter can be larger than the first inner diameter. In an embodiment, the second inner diameter can be disposed towards the tip 148 of gripping arm 146, relative to the first inner diameter, i.e. towards the tip 152 of the sheath 150. The sheath 150 can slidably engage the distal portion of the shaft 110 and a portion of the grip 140 along a longitudinal axis to transition the hub 130 between the open position and the closed position.

In the open position, an end portion 152 of the sheath 150 can be disposed proximally of the tip 148 of gripping arm 146, substantially adjacent the cross-beam 142. Further, the gripping arms 146 can be in a unstressed state, i.e. not elastically deformed. In the closed position, the end portion 152 of the sheath 150 can be advanced towards the tip 148 of gripping arm 146 to a position that is adjacent to, aligned with, or disposed beyond, the tip 148 of gripping arm 146. Further, the gripping arms 146 can elastically deformed radially inwards towards a central longitudinal axis, i.e. in a stressed state.

In the closed position, the outer surface 164 of the gripping arms 146 can be elastically deformed to align substantially parallel, i.e. aligned parallel with one of an inner surface of the sheath 150 and/or a central longitudinal axis 82. As such, an inner surface 162 of the gripping arms 146 can be angled relative to a central longitudinal axis extending from the second diameter (d2) adjacent the cross-beam 142 to a fifth diameter (d5) adjacent the tip 148 of gripping arm 146, which is less than second diameter (d2). In an embodiment, the fifth diameter (d5) is equal to or less than an outer diameter of the tip portion 28 of the catheter 10. As such, a portion of the inner surface 162 of the gripping arms 146 can grip a portion of the catheter 10 disposed therebetween.

In an embodiment, the inner surface 162 of the gripping arms 146 can include one or more gripping features such as ridges, barbs, protrusions, textured surfaces, one or more second materials having an increased frictional co-efficiency, or the like. The gripping features can further secure the tip portion 28 between the gripping arms 146 when the hub 130 is in the locked position. As will be appreciated, sliding the sheath 150 longitudinally from the locked position to the unlocked position can allow the gripping arms 146 to return to the elastically undeformed shape (FIG. 4A), releasing the tip portion 28 and allowing the hub 130 to disengage the catheter 10. In an embodiment, an end surface 136 of the gripping arm 146 can be angled relative to the central longitudinal axis to define a beveled entrance to the hub 130 and facilitate directing the tip portion 28 into the hub 130.

Advantageously, the gripping arms 146 can engage the catheter 10 at a point that is spaced apart from the delicate tip structures, thereby mitigating any damage to these structures when engaged with the catheter. Further, these delicate tip structures can be disposed within grip 140 and the sheath 150, protecting these tip structures from damage during subcutaneous tunneling.

In an embodiment, the movement of the sheath 150 along the longitudinal axis between the open position and the closed position can be actuated by the actuator mechanism 122. In an embodiment, the actuator mechanism can include mechanical, electrical, electro- mechanical, hydraulic, pneumatic mechanisms, or combinations thereof to slide the sheath 150 longitudinally between the open and closed positions.

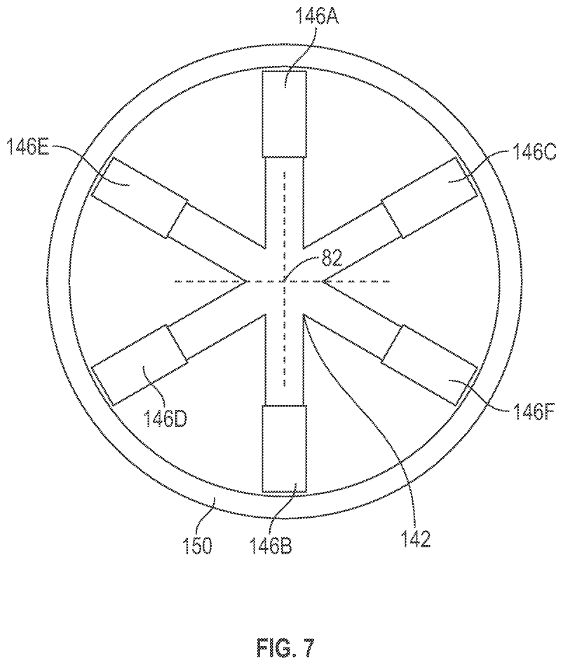

In an embodiment, as shown in FIG. 7, the grip 140 can include two or more gripping arms 146 arranged radially about the central longitudinal axis. For example, as shown, the grip 140 can include three pairs of gripping arms 146 arranged radially with a first gripping arm 146A disposed opposite a second gripping arm 146B, a third gripping arm 146C disposed opposite a fourth gripping arm 146D, and a fifth gripping arm 146E disposed opposite a sixth gripping arm 146F, across a central longitudinal axis 82. It will be appreciated, however, that other numbers and configurations of gripping arms 146 are also contemplated including odd numbers of gripping arms 146, even numbers of gripping arms 146, regularly radially arranged gripping arms (FIG. 7), or irregularly radially arranged gripping arms 146 where a first gripping arm is not disposed opposite a second gripping arm across the central longitudinal axis 82. Sliding the sheath 150 longitudinally can elastically deflect the one or more gripping arms 146 radially inwards towards the central longitudinal axis 82 to the locked position, as described herein.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A tunneler configured to place a portion of a catheter within a subcutaneous tunnel, comprising:
a shaft extending along a central longitudinal axis, and having a hub disposed at a first end and comprising:
a grip coupled to an end portion of the shaft and including a cross-beam supporting a first gripping arm and a second gripping arm, each of the first gripping arm and the second gripping arm transitionable between an unlocked position and a locked position; and
a sheath extending annularly about a portion of the grip and slidably engaged therewith along the central longitudinal axis to transition the first gripping arm and the second gripping arm between the unlocked position and the locked position.

2. The tunneler according to claim 1, wherein the first gripping arm extends longitudinally from a first end of the cross-beam and the second gripping arm extends longitudinally from a second end of the cross-beam, opposite the first end of the cross-beam, the cross-beam extending perpendicular to the central longitudinal axis.

3. The tunneler according to claim 1, wherein each of the first gripping arm and the second gripping arm includes an inner surface and an outer surface, in the unlocked position the outer surface is angled relative to the central longitudinal axis and the inner surface extends parallel to the central longitudinal axis, and in the locked position the inner surface is angled relative to the central longitudinal axis and the outer surface extends parallel to the central longitudinal axis.

4. The tunneler according to claim 3, wherein in the unlocked position the inner surfaces of the first gripping arm and the second gripping arm define an inner diameter (d2) that is equal to, or greater than, an outer diameter of the catheter.

5. The tunneler according to claim 3, wherein in the unlocked position the outer surface of each of the first gripping arm and the second gripping arm extends from an outer diameter (d3) of the cross-beam to an outer diameter (d4) of a tip of the gripping arm, the outer diameter (d4) at the tip of the gripping arm being larger than the outer diameter (d3) of the cross-beam.

6. The tunneler according to claim 5, wherein an inner diameter of the sheath is equal to, or greater than the outer diameter (d3) of the cross-beam and is less than the outer diameter (d4) of the tip of the gripping arm.

7. The tunneler according to claim 4, wherein in the locked position the inner surface of each of the first gripping arm and the second gripping arm extends from an inner diameter (d2) adjacent the cross-beam, to an inner diameter (d5) adjacent the tip of the gripping that is less than the outer diameter of the catheter.

8. The tunneler according to claim 1, wherein one or both of the first gripping arm and the second gripping arm includes a beveled surface adjacent a tip thereof and angled radially inwards towards the central longitudinal axis.

9. The tunneler according to claim 1, wherein one of the first gripping arm and the second gripping arm is biased towards the unlocked position.

10. The tunneler according to claim 1, wherein the grip is formed of a substantially rigid, or resilient, material such as a plastic, polymer, metal, alloy, or composite.

11. The tunneler according to claim 1, further including a handle disposed at a second end of the shaft, the handle including an actuator configured to slide the sheath longitudinally between a first position and a second position and transition the hub between a locked position and an unlocked position.

12. The tunneler according to claim 2, wherein the grip further includes a first shaft arm extending longitudinally from the first end of the cross-beam, and a second shaft arm extending longitudinally from the second end of the cross-beam, the first shaft arm and the second shaft arm engaging the end portion of the shaft in one of a press-fit, snap-fit, or interference fit engagement.

13. The tunneler according to claim 1, wherein the grip further includes a third gripping arm disposed opposite a fourth gripping arm across the central longitudinal axis, each of the third gripping arm and the fourth gripping arm extending longitudinally and transitionable between an unlocked position and a locked position.

14-18. (canceled)

19. A method of making a tunneler for placing a portion of a catheter within a subcutaneous tunnel, comprising:
forming a shaft extending along a central longitudinal axis;
coupling a hub at a first end of the shaft, comprising:
coupling a grip to an end portion of the shaft, the grip including a cross-beam supporting a first gripping arm and a second gripping arm, each transitionable between an unlocked position and a locked position; and
slidably engaging a sheath with a portion of the grip, the sheath transitionable between a first position and a second position to transition the first gripping arm and the second gripping arm between the unlocked position and the locked position.

20. The method according to claim 19, wherein the first gripping arm extends longitudinally from a first end of the cross-beam and the second gripping arm extends longitudinally from a second end of the cross-beam, opposite the first end of the cross-beam, the cross-beam extending perpendicular to the central longitudinal axis.

21. The method according to claim 19, wherein each of the first gripping arm and the second gripping arm includes an inner surface and an outer surface, in the unlocked position the outer surface is angled relative to the central longitudinal axis and the inner surface extends parallel to the central longitudinal axis, and in the locked position the inner surface is angled relative to the central longitudinal axis and the outer surface extends parallel to the central longitudinal axis.

22. The method according to claim 21, wherein in the unlocked position the inner surfaces of the first gripping arm and the second gripping arm define an inner diameter (d2) that is equal to, or greater than, an outer diameter of the portion of the catheter.

23. The method according to claim 21, wherein in the unlocked position the outer surface of each of the first gripping arm and the second gripping arm extends from an outer diameter (d3) of the cross-beam to an outer diameter (d4) at a tip of the gripping arms, the outer diameter (d4) at the tip of the gripping arms being larger than the outer diameter (d3) of the cross-beam.

24. The method according to claim 23, wherein an inner diameter of the sheath is equal to, or greater than, the outer diameter (d3) of the cross-beam and is less than the outer diameter (d4) of the tip of the gripping arms.

25. The method according to claim 22, wherein in the locked position the inner surface of each of the first gripping arm and the second gripping arm extends from an inner diameter (d2) adjacent the cross-beam, to an inner diameter (d5) adjacent the tip of the gripping arms that is less than the outer diameter of the portion of the catheter.

26. The method according to claim 19, wherein one or both of the first gripping arm and the second gripping arm includes a beveled surface adjacent a tip thereof and angled radially inwards towards the central longitudinal axis.

27. The method according to claim 19, wherein one of the first gripping arm and the second gripping arm is biased towards the unlocked position.

28. The method according to claim 19, wherein the grip is formed of a substantially rigid, or resilient, material such as a plastic, polymer, metal, alloy, or composite.

29. The method according to claim 19, further including a handle disposed at a second end of the shaft and including an actuator configured to slide the sheath longitudinally between the first position and the second position and transition the hub between a locked position and an unlocked position.

30. The method according to claim 19, wherein the grip further includes a first shaft arm extending longitudinally from the first end of the cross-beam, and a second shaft arm extending proximally from the second end of the cross-beam, the first shaft arm and the second shaft arm engaging the end portion of the shaft in one of a press-fit, snap-fit, or interference fit engagement.

31. The method according to claim 19, wherein the grip further includes a third gripping arm disposed opposite a fourth gripping arm across the central longitudinal axis, each of the third gripping arm and the fourth gripping arm extending longitudinally and transitionable between an unlocked position and a locked position.
